(19) United States
(12) Patent Application Publication
(10) Pub. No.: US 2003/0170878 A1
(43) Pub. Date: Sep. 11, 2003

(54) Method and apparatus for the treatment of biological suspensions

(76) Inventor: Anthony Dickson, New South Wales (AU)

Correspondence Address: GREENBLUM & BERNSTEIN, P.L.C., 1950 ROLAND CLARKE PLACE, RESTON, VA, 20191, US

(21) Appl. No.: 10/258,602

(22) PCT Filed: May 25, 2001
(86) PCT No.: PCT/AU01/00602

(30) Foreign Application Priority Data
May 25, 2000 (AU) PQ 7749

(51) Int. Cl. C12N 1/00

(52) U.S. Cl. 435/262.5; 435/243

(57) ABSTRACT

Method and apparatus for treating biological suspensions. A treatment agent is added to the biological suspension to form a mixture. The mixture is mechanically treated in a disintegrator 18 to homogenise and finely divide the mixture. The mixture is subsequently pressurised.

### FIELD OF THE INVENTION

This invention relates to a method and apparatus for the treatment of biological suspensions. As used in the specification the term “biological suspension” is intended to refer to any system in which organic materials are mixed with a liquid. It is not intended to be limited to mixtures in which the organic material is suspended but includes mixtures in which the organic material entirely or partially floats or precipitates. The term is also intended to systems in which the amount of organic material is relatively small compared to the liquid such as for example drinking water prior to treatment.

### BACKGROUND OF THE INVENTION

The method and apparatus of the invention are applicable to a wide range of processes in particular the disinfection and solids oxidation of biological suspensions. The biological suspensions include water related products such as sewerage, aquaculture, drinking water, wash down water from wineries or food and chemical processors. Although the invention will be primarily described in relation to its application to the treatment of sewerage it is to be understood that the invention has application to these various areas. Reference to existing processes and apparatus in the specification are included only for the purposes of exemplification and are not to be construed as statements of the common general knowledge in the field.

Treatment of sewerage has included sewerage treatment plants using a three stage process. A primary treatment involves physical separation of materials and a screening process. Secondary treatment involves biological reduction processes and further physical separation. This is followed by a tertiary treatment involves physical chemical or biological processes and subsequent filtration and ponding. The difficulties associated with this form of treatment include the physical size of the treatment plant, the environmental impact and long treatment times. More particularly, treatment plants can include large ponding areas open to the atmosphere. Not only do these ponding areas occupy a large amount of space but they also produce unwanted unpleasant odours and have the potential for environmental contamination. The inherent nature of the processes used in such treatment plants can involve retention of the sewerage of the various processing steps for up to 30 days.

### DISCLOSURE OF THE INVENTION

It is an object of this invention to provide a method and apparatus for the treatment of biological suspensions which will overcome or at least ameliorate one or more of the foregoing disadvantages.

Accordingly, in a first aspect this invention provides a method of treating a biological suspensions including the steps of

(a) adding a treatment agent to the biological suspension to form a mixture;

(b) mechanically treating the mixture to homogenise and finely divide the mixture; and

(c) subsequently pressurising the mixture.

In another aspect this invention provides an apparatus for treating a biological suspension including means for mechanically treating a mixture of the biological suspension and a treatment agent to homogenise and finely divide the mixture, and means to subsequently pressurise the mixture.

The mixture is preferably pressurised for a predetermined period of time.

The invention has particular application to the treatment of sewerage to kill or destroy bacteria, pathogens and viruses. In this application the treatment agent is preferably an oxidising agent. Suitable oxidising agents are ozone or chlorine based compounds and include hypochlorous acid (HOCl) and any compound with disinfecting or oxidising properties. As will be known to those skilled in the art hypochlorous acid can be provided by the addition of sodium hypochlorite (NaOCl) to water. Since the systems being treated usually contain water, in practice sodium hypochlorite can be added directly.

In a preferred form of the invention the mechanical treatment includes passing the mixture through a high speed disintegrator. The disintegrator preferably subjects the mixture to a high shear and/or fluctuating pressures. The disintegrator preferably substantially emulsifies the mixture. The mechanical treatment preferably results in the particle size of most of the mixture being less than about 30 microns. More preferably substantially all of the particles in the mixture have a size less than about 10 microns. Even more preferably, substantially all of the particles have a size in the range of 1 to 5 microns.

The fine division of the mixture is believed to improve the access of the treatment agent to the components of the biological suspension. More particularly, it is thought that in the case of sewerage the mechanical treatment reduces the “hiding places” within the material for faecal coliform, pathogens and viruses so that the treatment agent is effective or at least the treatment time is reduced. In this regard the strong oxidising ability of many treatment agents can in time break down the biological material or at least its structure and kill and destroy the coliforms, pathogens and viruses distributed within the material. However, in practice this requires unacceptable long treatment times. The present invention enables the treatment times to be reduced to commercially attractive levels. The mechanical treatment is also thought to damage or lyse at least some of the cells to provide improved access for the treatment agent.

The mixture is preferably pressurised to a pressure greater than about 500 kPa. More preferably the pressure is about 800 kPa. The pressure used and treatment time required are interdependent and can be adjusted to suit particular applications. By way of example a treatment pressure of 600 kPa has been found to require a treatment time of about 30 minutes in order to reduce the amount of FC and FS to levels undetectable in conventional analysis procedures.

The pressurisation step is preferably carried out in a cell diffusion unit. In one embodiment this unit is formed by a long continuous conduit or tube in which the sewerage is maintained under pressure.

After pressurisation the sewerage is preferably filtered. This can involve treatment in a flocculation chamber. For example this chamber can be used to mix a polyelectrolyte with the sewerage to combine all the suspended solids. The suspended solids can then be removed for example using a continuous micro filter. The micro filter preferably can in one embodiment take the form of a continuous belt inclined upwardly. As the sewerage travels up an inclined slope on the filter bed the water seeps through the belt and the solids remain. The solids are subsequently scraped from the belt. It will be appreciated that other filtration systems or combinations of filtration systems can be used. These include sand filters and membrane filters.

Prior to treatment the sewerage preferably passes through a pH correction tank to appropriately adjust the acidity. The correct amount of treatment agent, such as hypochlorous acid is preferably adjusted under computer control.

The entire system is preferably enclosed and operated under computer control.

The advantages of the method and apparatus according to the present invention include:

(i) the entire treatment system is enclosed eliminating odours and reducing health risks

(ii) it does not require any bacterial colonies

(iii) it is currently believed that the invention can be constructed in a plant approximately 10 times smaller than other plants for the same treatment volume

(iv) the method and apparatus of this invention are unaffected by temperature

(v) unlike treatment plants involving biological processes there is no time delay in starting the plant

(vi) the method and apparatus of the present invention are able to be readily adjusted to treat erratic input surges of sewerage and provide consistent safe outputs

(vii) the method and apparatus of this invention at least in the preferred embodiment do not require highly skilled operators

(viii) the present invention provides a plant that can be operated by computers on site or from a remote location via modems or radio links

(ix) the present invention provides a plant that is more efficient and consequently more cost effective than other treatment plants

(x) the high degree of removal of unwanted contaminants results in the solids becoming a valuable and safe fertiliser

(xi) the treated effluent is completely safe for use in various applications

(xii) the method and apparatus of this invention have been found to kill up to 95 to 100% of viruses, pathogens and bacteria in sewerage.

One embodiment of the invention will now be described, by way of example only, with reference to the drawings.

### BRIEF DESCRIPTION OF THE DRAWINGS

FIG. 1 is a schematic diagram of a sewerage treatment plant incorporating the method and apparatus of the present invention;

FIG. 2 is a schematic drawing more detail of part of the treatment plant shown in FIG. 1;

FIG. 3 is an enlarged view of the high velocity disintegrator shown in FIGS. 1 and 2;

FIG. 4 is a plan view and sectioned elevation view of an inner stator forming part of the high velocity disintegrator shown in FIG. 3;

FIG. 5 is a plan view and sectioned elevation view of an outer stator forming part of the high velocity disintegrator shown in FIG. 3;

FIG. 6 is a plan view and sectioned elevation view of a rotor forming part of the high velocity disintegrator shown in FIG. 3; and

FIG. 7 is a perspective view of a dispersion disc forming part of the high velocity disintegrator shown in FIG. 3.

### BEST MODE FOR CARRYING OUT THE INVENTION

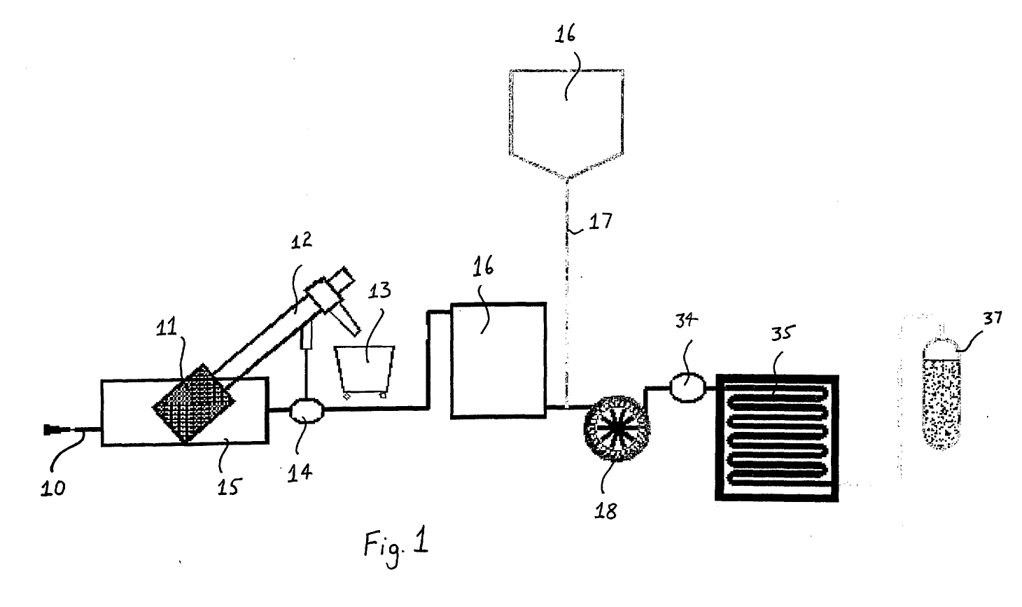

FIG. 1 shows the general layout of a sewerage treatment plant incorporating the method and apparatus of the present invention. Some of the components in the treatment plant are known in the art and a detailed description is not required for an appreciation of the present invention.

The sewerage or influent entering at 10 initially passes through a primary screen and primary maceration unit 11 of a type known to those skilled in the art. The foreign matter is separated and routed by a conveyor 12 to a collection system 13. The influent is directed to a flow control device 14 of known type provided to control the rate of feed of influent to the remainder of the treatment system. Excess volume is directed through a holding facility 15 and returned to the input in the known manner.

Influent passes to pH correction unit 16. This unit 16 includes a pH correction tank in which the acidity of the sewerage is adjusted to the desired pH input level. Following this adjustment the sewerage is injected with the correct amount of hypochlorous acid required for the process by supplying sodium hypochlorite from storage tank 16 via line 17. The correct amount is preferably determined by computer monitoring of the solid oxidation in the cell diffusion unit 35 further downstream.

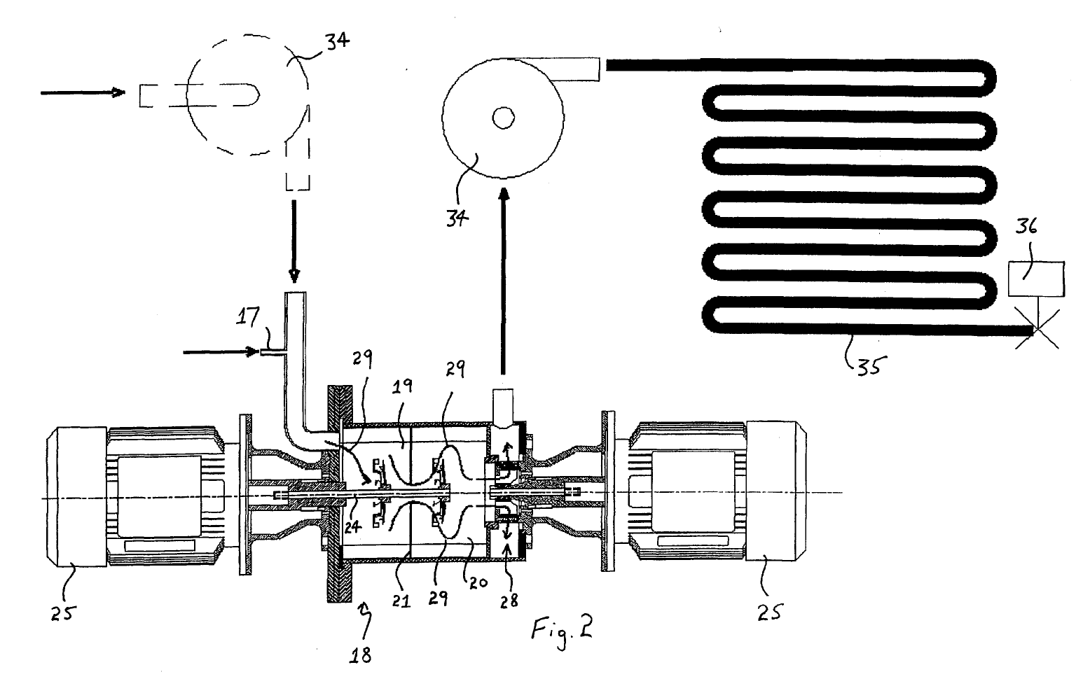

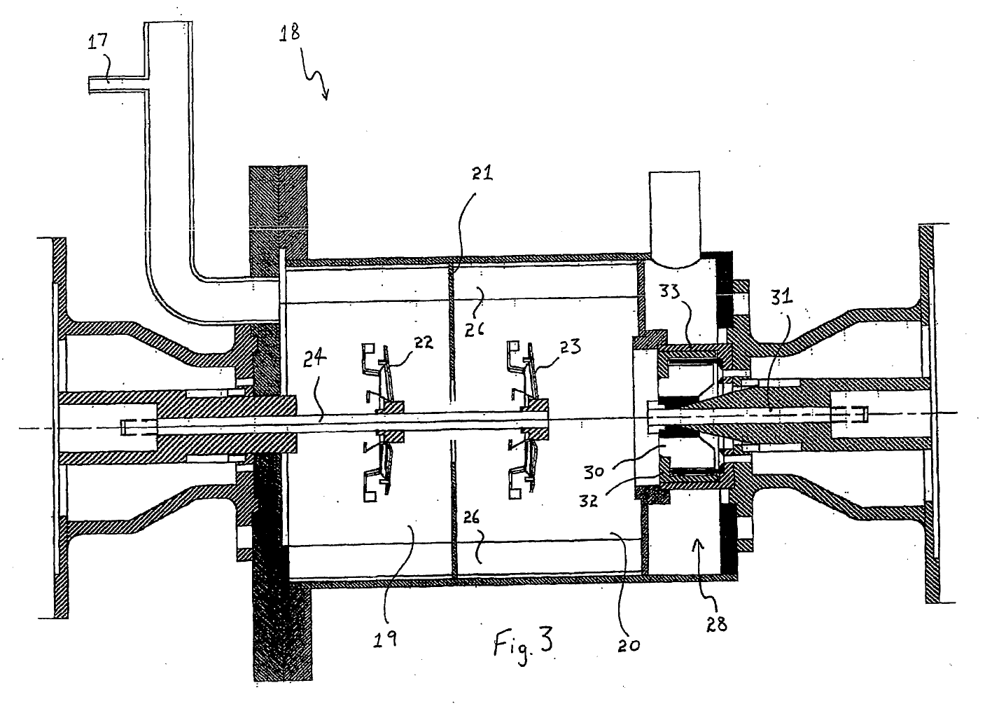

After addition of the sodium hypochlorite the influent flows to a high velocity pressure disintegrator 18. The disintegrator 18 is also shown in FIGS. 2 and 3. The disintegrator has a central region divided into two compartments 19, 20 by a compartment baffle 21. Dispersion disks 22, 23 are mounted on a disperser shaft 24 respectively in the two compartments 19, 20 formed by the compartment baffle 21. An electric motor 25 is provided to drive the disperser shaft 24 and hence dispersion discs 22, 23 at high rotational speed. Baffles 26 are provided circumferentially around the internal perimeter of the compartments 19, 20. An inlet 27 is provided to the first of the compartments 19. The mixture passes through the two compartments 19, 20 in the high velocity disintegrator 18 where it is subjected to a mechanical treatment involving high shear and fluctuating pressures as a result of the rotating dispersion discs 22, 23.

After treatment by the dispersion discs 22, 23 the mixture flows to a rotary compressor 28. Arrows 29 in FIG. 2 illustrate the flow of the mixture. Rotary compressor 28 includes a rotor 30 mounted on a compressor shaft 31 located inside two stators 32, 33 as described in detail below. Shaft 31 is driven by an electric motor 25 to rotate rotor at high speed. The mixture is forced outwardly through the stators 32, 33 as indicated by arrows 29 which again subjects the mixture to high shear and/or fluctuating pressure. This results in the mixture of sewerage and acid being homogenised and the particles being finely divided. In a preferred operation of the device most of the particles have a size in the range of 1 and 5 microns.

From the rotary compressor 28 the mixture is fed to a high-pressure pump 34. The high-pressure pump 34 maintains a pressure of about 600 kPa in the mixture downstream of the pump. 34 The mixture is pumped under this pressure through a cell diffusion unit 35. The cell diffusion unit 35 is a series of tubes linked end to end to produce a long continuous path. A restriction value 36 (FIG. 2) can be provided to maintain the desired pressure. The sewerage is under pressure long enough to force the hypochlorous acid into the cells via osmosis. Typically the mixture is maintained under pressure about 30 minutes. Hypochlorous acid is a most effective germicide. It is known in the industry as the free available chlorine residual. Germicidal efficiency of hypochlorous acid is due to the relative ease with which it can penetrate cell walls. This penetration is comparable to that of water and can be attributed to its low molecular weight and its electrical neutrality. The hypochlorous acid also has the ability to reduce the suspended cells, nitrogen and phosphorous.

From the cell diffusion unit the mixture passes through a filtration system 37. A number of filtration systems are suitable including advanced coaggulation systems, flocculation systems and reverse osmosis systems. In one form of the invention the microfilter (not shown) operates at the end of the flocculation chamber. The microfilter takes the form of a continuous belt filter somewhat similar in appearance to a conveyor belt. It is inclined upwardly and the sewerage travels up the slope on the filter belt. The water seeps through the belt and the solids remain on the belt. When the solids reach the top of the filter they are dry enough to be scraped into a container for packaging.

The water that is separated by the filtration process is inert and suitable for use in a variety of applications.

The entire process can be conducted under computer control.

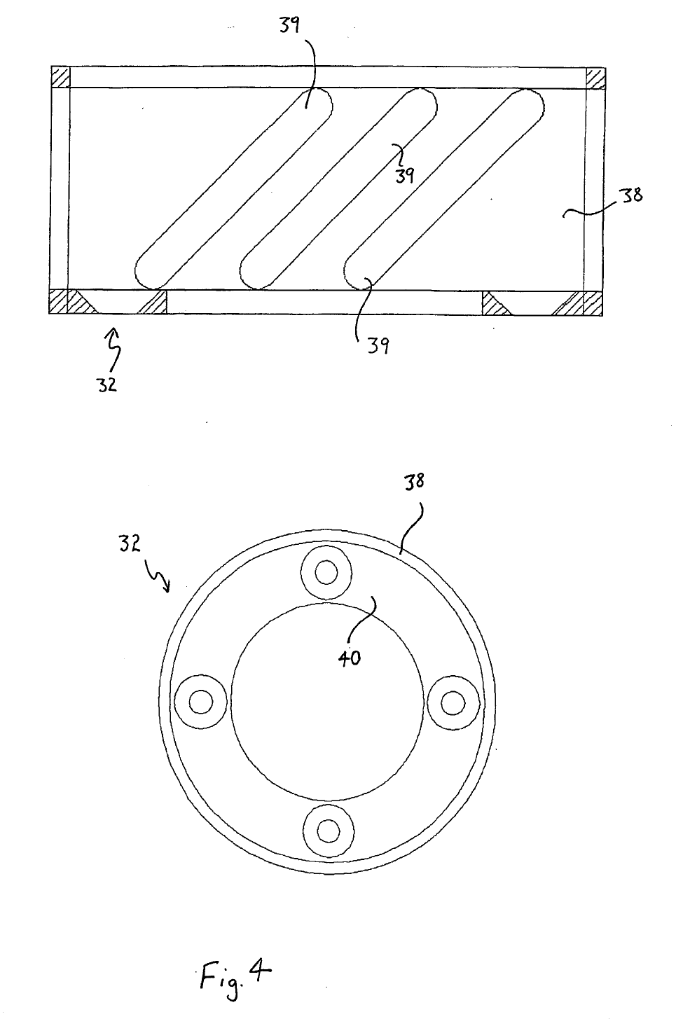

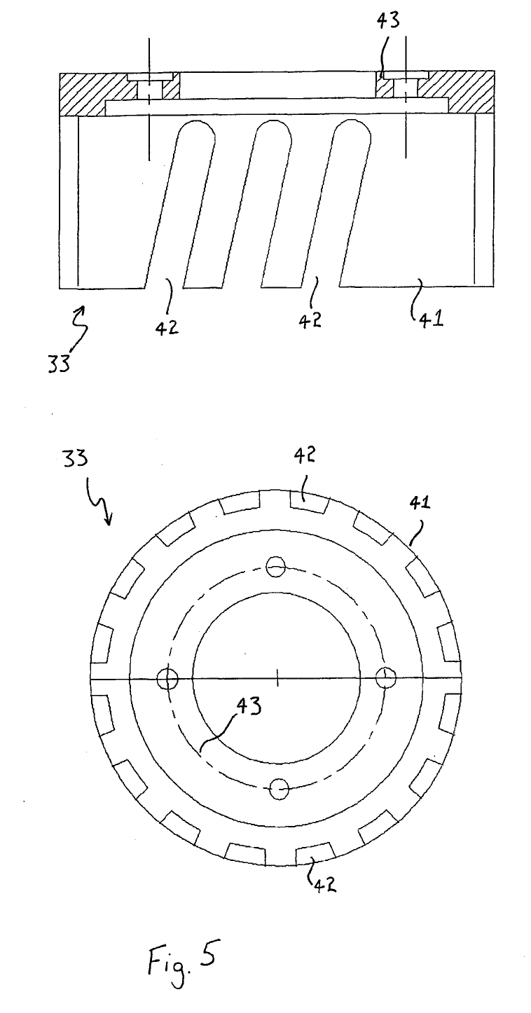

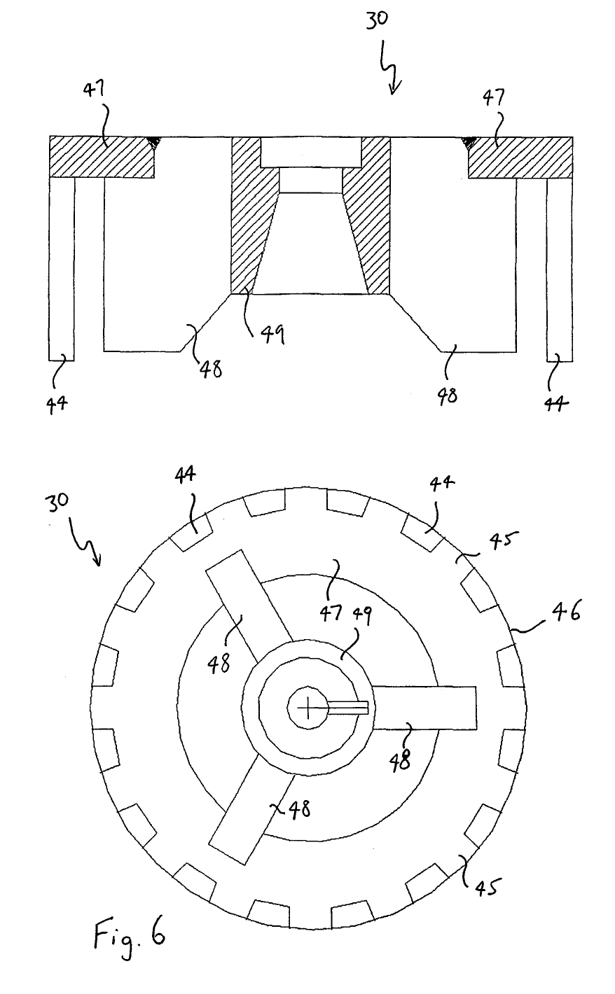

FIG. 4 shows the inner stator 32 used in the disintegrator 18 of FIG. 3. As seen in FIG. 3 the inner stator closely nests within the outer stator 33. Inner stator 32 has a generally cylindrical outer surface 38 in which angled through slots 39 are formed. A mounting flange 40 extends radially inwardly. The outer stator 33 shown in FIG. 5 is generally similar to the inner stator 32. It has a cylindrical outer surface 41 in which open ended through slots 42 are formed. A mounting flange 43 extends radially inwardly. It will be apparent that the two stators 32, 33 are fixedly mounted within the disintegrator 18 and the outward passage of the mixture occurs by sequential passage through the two sets of slots 39, 42. FIG. 6 shows the rotor 30 that is mounted to shaft 31. The rotor has a series of spaced apart fingers 44 separated by spaces 45. The fingers 44 are arranged around the circumference of a cylindrical surface 46. A radial flange 47 extends inwardly and is supported by three mounting vanes 48 that connect with a collar 49. The collar 49 is attached to shaft 31. Rotor 30 is driven by electric motor 25 at high rotational speed. The passage of the mixture through spaces 45 and subsequently through slots 39 and 42 subjects the mixture to high shear and/oil fluctuating pressure.

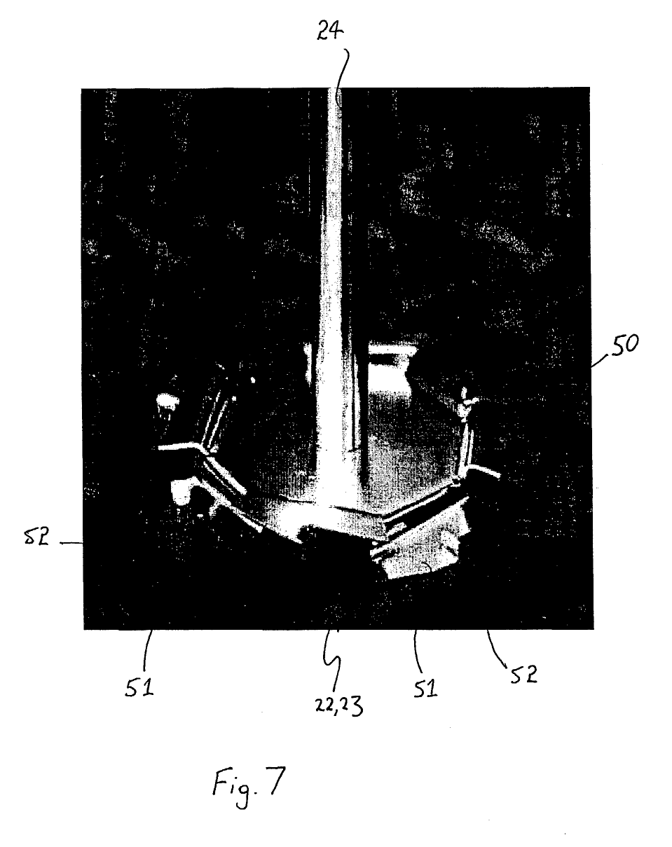

FIG. 7 shows one of the dispersion discs 22, 23 forming part of the disintegrator shown in FIG. 3 in greater detail. Dispersion disc 22, 23 is mounted on shaft 24. Dispersion disc 22, 23 is formed by a radially extending disc 50 on which agitating formations 51 or teeth are formed. The teeth 51 have a generally L-shaped profile with axially extending tabs 52. It will be apparent that when the disc 22, 23 is driven at high rotational speed considerable shear and or fluctuating pressure is caused in the mixture passing in the vicinity of the rotating disc 22, 23.

As shown in FIG. 2 the high pressure pump 34 (shown in dotted outline) can alternatively be located upstream of the high velocity disintegrator 18. This results in the mixture being pressurised during its passage through the disintegrator 18. In some applications this allows the overall pressurisation time to be substantially reduced. Further in some applications sufficient treatment can be achieved during passage through the disintegrator 18 and/or subsequent piping allowing the elimination of the cell diffusion unit 35.

The levels of faecal coliform (FC) E.coli and Salmonellae in samples from the inlet and outlet of the sewerage system described were measured. The results are shown in Table 1. It will be seen that the levels of faecal coliform and faecal streptococci at the outlet were below detectable levels. It has also been found that large reductions of suspended solids as well as the amount of a oil and grease in sewerage can be achieved.

TABLE 1

| | Thermotolerant Faecal | Faecal Streptococci |
|---|---|---|
| | Coliform (cfu/100 ml) | (cfu/100 ml) |
| Sample 1 | | |
| inlet | 3,200,000 | 960,000 |
| outlet | n.d. | n.d. |
| Sample 2 | | |
| inlet | 1,900,000 | 64,000 |
| outlet | n.d. | n.d. |
| Sample 3 | | |
| inlet | 1,100,000 | 685,000 |
| outlet | n.d. | n.d. |

n.d. = not detectable

The foregoing describes only one embodiment of the present invention and modifications can be made without department from the scope of the invention.

1. A method of treating a biological suspensions including the steps of
(a) adding a treatment agent to the biological suspension to form a mixture;
(b) mechanically treating the mixture to homogenise and finely divide the mixture; and
(c) subsequently pressurising the mixture.

2. A method as claimed in claim 1 wherein the mechanical treatment of the mixture includes the step of passing the mixture through a high speed disintegrator.

3. A method as claimed in claim 2 wherein the disintegrator substantially emulsifies the mixture.

4. A method as claimed in claim 2 or claim 3 wherein the high speed disintegrator subjects the mixture to high shear and fluctuating pressure.

5. A method as claimed in any one of claims 2 to 4 wherein the disintegrator includes two sequential stages one of said stages treating the mixture with at least one rotating dispersion disc and a second stage passing the mixture through a rotary compressor.

6. A method as claimed in any one of claims 1 to 5 wherein the mechanical treatment of the mixture reduces the particle size of substantially all of the mixture to less than about 30 microns.

7. A method as claimed in claim 6 wherein substantially all of the particles in the mixture have a size less than about 10 microns.

8. A method as claimed in claim 7 wherein substantially all of the particles in the mixture have a size in the range of 1 to 5 microns.

9. A method as claimed in any one of claims 1 to 8 wherein the mixture is pressurised to greater than about 500 kPa.

10. A method as claimed in any one of claims 1 to 8 wherein the mixture is pressurised to about 800 kPa.

11. A method as claimed in any one of claims 1 to 8 wherein the mixture is pressurised to about 600 kPa for at least 30 minutes.

12. A method as claimed in any one of claims 1 to 11 wherein the mixture is pressurised by being maintained under pressure as it passes through a long continuous conduit.

13. A method as claimed in any one of claims 1 to 10 wherein the treatment agent is an oxidising agent.

14. A method as claimed in claim 13 wherein the oxidising agent includes hypochlorous acid.

15. An apparatus for treating a biological suspension including means for mechanically treating a mixture of the biological suspension and a treatment agent to homogenise and finely divide the mixture, and means to subsequently pressurise the mixture.

16. An apparatus as claimed in claim 15 wherein means for mechanically treating the mixture includes a high speed disintegrator.

17. An apparatus as claimed in claim 16 wherein the disintegrator substantially emulsifies the mixture.

18. An apparatus as claimed in claim 15 or claim 16 wherein the high speed disintegrator subjects the mixture to high shear and fluctuating pressure.

19. An apparatus as claimed in any one of claims 16 to 18 wherein the disintegrator includes two sequential stages having at least one rotating dispersion disc and a second stage having a rotary compressor.

20. An apparatus as claimed in any one of claims 15 to 19 wherein the treatment of the mixture reduces the particle size of substantially all of the mixture to less than about 30 microns.

21. An apparatus as claimed in claim 20 wherein substantially all of the particles in the mixture have a size less than about 10 microns.

22. An apparatus as claimed in claim 21 wherein substantially all of the particles in the mixture have a size in the range of 1 to 5 microns.

23. An apparatus as claimed in any one of claims 15 to 22 wherein the mixture is pressurised to greater than about 500 kPa.

24. An apparatus as claimed in any one of claims 15 to 22 wherein the mixture is pressurised to about 800 kPa.

25. An apparatus as claimed in any one of claims 15 to 22 wherein the mixture is pressurised to about 600 kPa for at least 30 minutes.

26. An apparatus as claimed in any one of claims 15 to 25 wherein the mixture is maintained under pressure as it passes through a long continuous conduit.

27. An apparatus as claimed in any one of claims 15 to 26 wherein the mixture is treated with an oxidising agent.

28. An apparatus as claimed in claim 27 wherein the oxidising agent includes hypochlorous acid.
